(19) United States
(12) Patent Application Publication
(10) Pub. No.: US 2020/0258614 A1
(43) Pub. Date: Aug. 13, 2020

(54) Organizational Wellness Delivery and Tracking

(71) Applicant: OWA Holdings Ltd., London (GB)

(72) Inventor: Preetha Rama Bhagavaddasa, Shenoynagar (IN)

(21) Appl. No.: 16/787,383

(22) Filed: Feb. 11, 2020

(30) Foreign Application Priority Data
Feb. 11, 2019 (IN) 201911005317

(51) Int. Cl. G16H 20/70; G06Q 10/10; H04N 21/266

(52) U.S. Cl. CPC G16H 20/70; H04N 21/266; G06Q 10/1057

(57) ABSTRACT

Meditation instruction that is institutionalized with employees and organizational leaders offered a type of meditation based upon the type of stress encountered and collects metrics associated with the meditation instruction.

### CROSS REFERENCE TO RELATED APPLICATIONS

This application claims priority to India Patent Application No. 201911005317, filed Feb. 11, 2019, titled Organizational Wellness Delivery and Tracking, which application is incorporated by reference in this application in its entirety.

### TECHNICAL FIELD

The present invention relates generally to providing mental health care to an organization and more particularly to quantifying results of provided mental health care by delivering and providing meditation that is personalized to an organization and to every individual within the organization.

### BACKGROUND

Organizations, such as business, typically have numerous employees. These employees are often dissatisfied with the recognition and meaning they find with their jobs. But, dissatisfaction is often a mutual experience between employees and employers that affect productivity and creativity of the organization. Some examples of the causes of the mutual dissatisfaction are often attributed to: no harmony between teams, corporate politics, and disengagement of employees from their work. In a typical organization, only 13% of the employees are passionately engaged in their jobs. The other 87% are to some degree dissatisfied and less engaged in their jobs.

The dissatisfaction or disharmony in an organization may be caused by cerebral, mental, and emotional stress. The cerebral, mental, and emotional stress manifests in an organization as various problems among employee, teams, and management that leads to dissatisfaction. The three forms of stress within an individual, creates a disruptive or distracted individual and drives entire departments within an organization and eventually the entire organizations into stress. Stress problems keep surfacing in multiple guises as interpersonal friction, politics, and results in a lack of creativity and fatigue or indiscipline within the organization.

As long as the roots of the problems are not addressed, namely stress, the problems of dissatisfaction cannot be reduced and/or eliminated. Current approaches to reducing dissatisfaction have included offering greater benefits, salary hikes, promotions, vacations, work-out facilities, table top games (e.g., ping-pong tables) and etc. These approaches have proven to control the problem for a time, but not really address the problem at its root. But a problem that has occurred with these prior approaches is the inability to quantify an organizational impact.

What is needed is an approach that addresses the dissatisfaction within an organization while nourishing its culture and being able to quantify the impact on the organization.

### SUMMARY

An approach that addresses the dissatisfaction within an organization while nourishing its culture is disclosed herein. An organization and its culture are changed through the institutional implementation of meditations, which contribute to the emergence of a peaceful and a connected individual with reduced dissatisfaction. Meditations transform organizations leaders, while revolutionized the culture of the entire organizations. Consistently practicing these meditations results in greater levels of employee happiness and therefore greater engagement at work, resulting in increased productivity and profit of the company; therefore, both the employee and the employer are satisfied. The type of meditation provided is based upon the stress type and the delivery method is via streaming media.

Unlike traditional approaches of just making a program available, Meditation instruction is institutionalized with employees and organizational leaders use directed based upon the type of stress encountered enabling metrics on dissatisfaction to be collected and monitored.

Other devices, apparatus, systems, methods, features, and advantages of the invention will be or will become apparent to one with skill in the art upon examination of the following figures and detailed description. It is intended that all such additional systems, methods, features, and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 depicts a computer client being provided with media streamed from a streaming server in accordance with an example implementation of the invention.

FIG. 2 depicts a computer client graphical user interface for selecting the media of FIG. 1 based upon a type of stress.

FIG. 3 depicts an administration graphical user interface for managing the user in an organization that has access to the server and media of FIG. 1 in accordance with an example implementation.

FIG. 4 is a flow diagram of the user of the system depicted in FIGS. 1-3.

### DETAILED DESCRIPTION

The present invention relates to an approach for meditation instruction that is institutionalized with employees and organizational leaders and that is offered and delivered in different meditation instruction based upon the type of stress encountered. The present invention further collects metrics associated with the meditation instruction described.

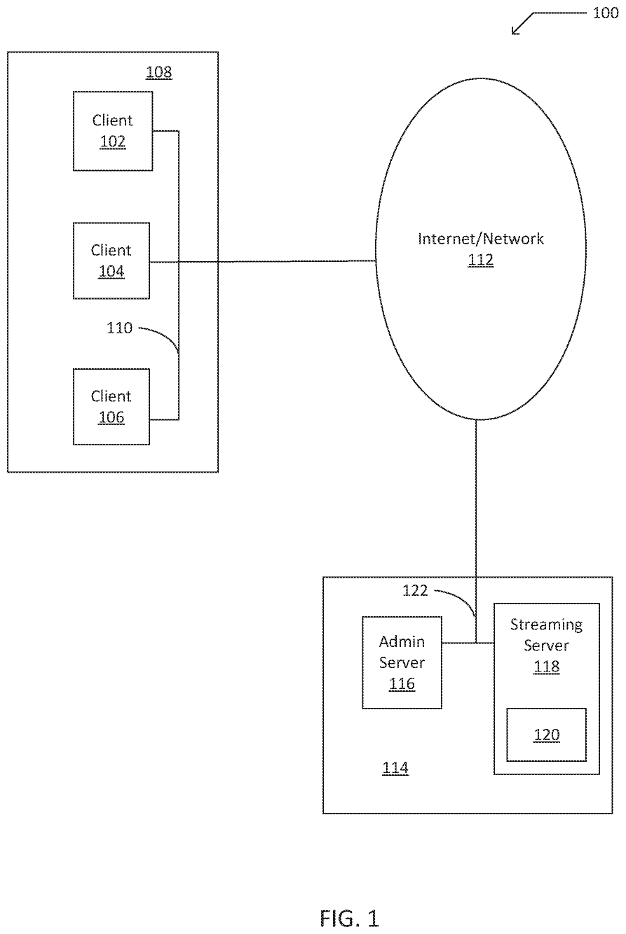

In FIG. 1, a diagram 100 of computer clients 102-106 being provided with media 120 streamed from a streaming server 118 in accordance with an example implementation of the invention is depicted. A computer client, such as 102 may be a workstation, personal computer, tablet, smartphone, or other processor-controlled devices that are capable of receiving the media stream and playing it for an employee/user or for a group of employees/users that come together to meditate. The meditation for each individual or group of individuals may be selected based upon the type of meditation best suited for the individual in the groups based upon the collective needs of the individuals in the group or common sources of stress.

The computer client 102 may be implemented in hardware or software implemented to run on a processor controlled device. Typically, a client is implemented to run in an operating system (Windows, Linux, IOS, etc. . . . ) on a processor-controlled device. The computer clients 102-106 are typically located in a business 108, campus, or organization connected by one or more local area networks 110. The local area networks 110 typically have a connection to a wide area network and eventually the internet 112. The local area networks may be implemented as wired, wireless, or a combination of wired and wireless network cable and devices.

An enterprise meditation provider 114 is depicted with an administration server 116 and streaming server 118 connected by local area network 122. In practice, multiple administration servers 116 and streaming servers 118 may be deployed by the enterprise meditation provider 114. In other implementations, the administration server 116 and streaming server 118 may be implemented remotely on servers in a server farm, such as run by AMAZON.

The streaming server 118 has access to a meditation media files 120 and when selected by a client, digitally streams the selected media to the requesting client. The digital media may be encoded as MP4, AVI, DVIX, or other audio-video encoding approach. In other implementations, the streaming server 118 may communicate with a remote storage location to access and stream media to clients.

In other implantations, encryption may also be used to secure the streaming media with decryption capabilities located in or near the clients 102-106. Other types of media security may also be employed, such as limiting media to be streamed to predetermined network locations/LANs based on internet addresses. In other implementations, other or additional security may be employed. If medial information is solicited from users/employees additional security for that data will be employed to meet government privacy laws and requirements, such as Health Insurance Portability and Accountability Act of 1996 in the United States.

The administration server 116 manages user accounts, licenses, survey data, and other metrics. Report generators that process the data are typically located with the administration server 116. In some implementations, electronic messaging capability may also be implemented or controlled from the administration server 116. The messaging may be text messaging, email, or other types of electronic communication. The communication provides reminders to the user, reports on progress, and in some implementations two-factor authentication for accessing media.

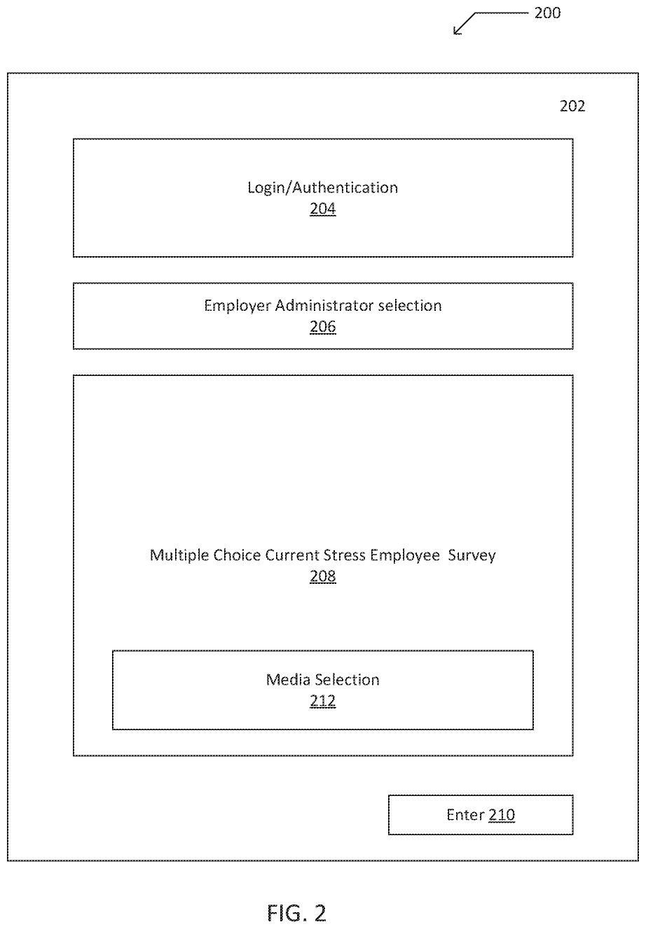

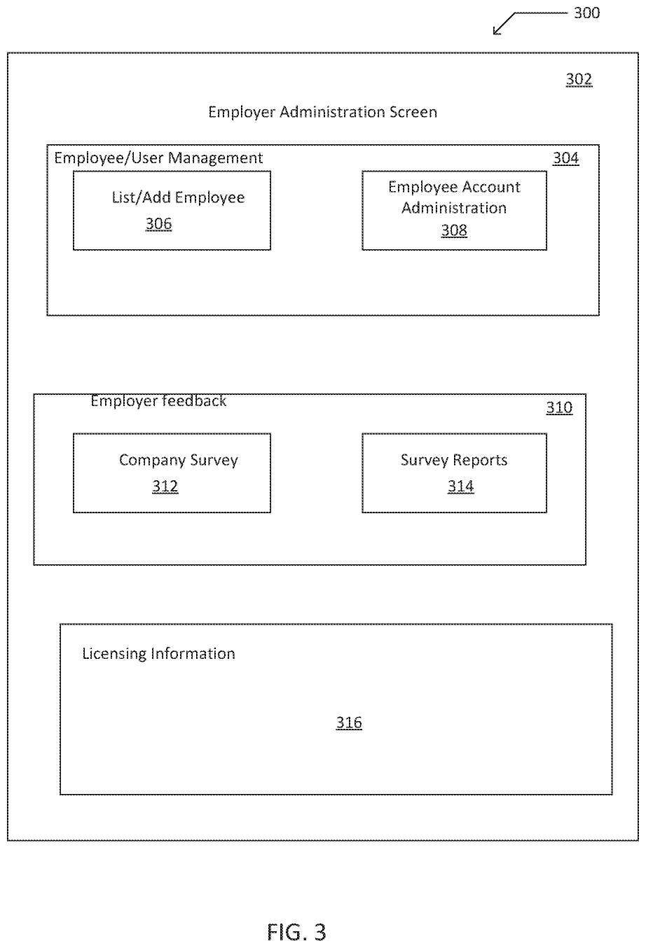

Turning to FIG. 2, a drawing 200 of a client graphical user interface 202 for selecting the media 120 of FIG. 1 based upon a type of stress. A user logs in via a login/authorization 204 portion of the screen. The login may employ two-factor authentication that is sent to an employee's work email. This achieves a double security check that the user is an employee and that it is the person logging in. If the user is an employer administration, they are presented with additional options for managing users and licenses 206 and may access the graphical user interface depicted 300 in FIG. 3. However, the management of user licenses and data may be collected and managed by third parties for security purposes, providing only non-confidential data or non-personal data to the employer or administrator.

Typically all users of the meditation media are provided with a multiple choice current stress employee survey 208 to determine what type of meditation is required or best suited for each individual employee. The survey may be provided periodically or during every login. The data from the survey may be stored by the administration server 116 for further processing and reporting. Upon completion of the multiple choice current stress employee survey 208, the user is presented with media selections 212 for streaming from the media server 118, or alternatively, provided with options to schedule meditation in a group setting with other employees at a designated location arranged by the employer.

In other implementations, different types of surveys may be provided for different types or categories of employees. In yet other implementations, additional periodic surveys may be provided to all employees to collected organizational data regarding stress. In still yet other implementations, additional surveys may be executed or otherwise given on behalf of the employer focused on other topics of interest to the company.

For example, the surveys can help identify specific areas of struggle for employees by asking the employees different tailored questions and can offer meditation choices that will target the identified concerns or problems. The survey may, for example, determine that an employee is not sleeping well, suffers from low energy levels, is having trouble focusing and/or is experiencing anxiety. The meditation sessions offered can then be those designed to address the identified areas of concern. Subsequent surveys, tracking and reporting can help determine the success of the different meditations on employees. Companies can then require participation or offer incentives for employees to participate to improve the overall health and well-being of their employees, and track improvement in areas of struggle.

In FIG. 3, a diagram 300 of an administration graphical user interface 302 for managing the users in an organization that has access to the servers and media of FIG. 1 in accordance with an example implementation. With Employee/User Management 304 an employer administrator may list or add employees with the list/add employee graphical user interface button 306. The employer administrator may also manage employee user accounts using graphical user interface button 308. Again, optionally, accounts may be outsourced and the confidentiality of certain personal information collected by the surveys may be maintained.

The employer administrator may issue predefined surveys associated with meditation or define their own surveys by selecting the company graphical user interface survey button 312 in the employer feedback 310 section of the graphical user interface. Results from surveys and report processing are provided by selecting the Survey Reports graphical user interface button 314, where additional windows are presented for report generation and survey processing. The employer administrator also has the ability to verify licensing information, renew licenses, or make changes in licenses in the licensing information section 316 of the employer administration screen graphical user interface 302.

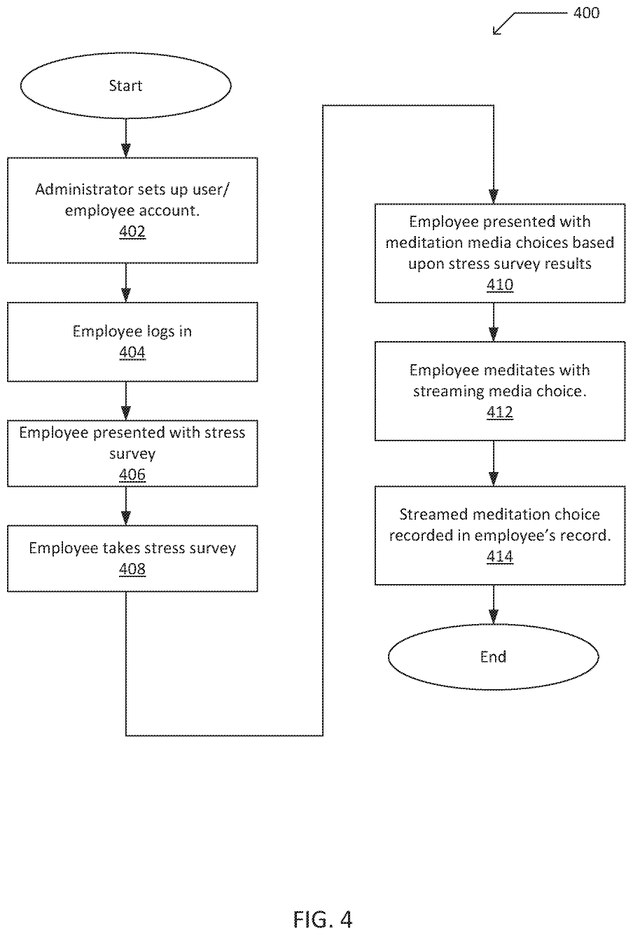

Turning to FIG. 4, a flow diagram 400 of the user of the system depicted in FIGS. 1-3. In one example of an implementation of the invention, an employer administrator creates or sets up a user/employee account 402. The account in the current embodiment is located on the administration server 116. An employee is then able to log in 404 to the meditation system using a client 102. In other implementations, two-factor authentication or other security approaches may be used to verify the user. The employer/user is presented with a meditation stress survey 406. The stress survey may be presented each time a user logs in the current implementation. In other implementations, the stress survey may be provided periodically.

The employee/user completes the stress survey 408 and is presented with meditation media choices based upon the stress survey 410. The employee/user then chooses the meditation that they wish to do 412. The streaming server 118 then streams the selected meditation media 120 to the client 102 used by the employee/user. Optional, the employee or user selects a time and location to attend a group meditation. Optionally, the meditation choices may only be offered at certain times to avoid interfering with work regardless where the server streams the selected meditation. In either case, the streamed media may be encrypted or otherwise secured for transmission between the streaming server 118 and client 102. The user viewing of the meditation media is then recorded in a user record accessed by the administration server 116. The user records including account information may be stored in a database, such as SAS. In other implementations, a record of viewed media may be created and an employer charged for the amount of delivered media in addition to a general licensing scheme.

It will be understood and is appreciated by persons skilled in the art, that one or more processes, sub-processes, or process steps described in connection with FIG. 4 may be performed by hardware and/or software (machine-readable instructions). If the approach is performed by software, the software may reside in software memory in a suitable electronic processing component or system such as one or more of the functional components or modules schematically depicted in the figures.

The software in software memory may include an ordered listing of executable instructions for implementing logical functions (that is, “logic” that may be implemented either in digital form such as digital circuitry or source code or in analog form such as analog circuitry or an analog source such an analog electrical, sound or video signal), and may selectively be embodied in any computer-readable medium for use by or in connection with an instruction execution system, apparatus, or device, such as a computer-based system, processor containing system, or other system that may selectively fetch the instructions from the instruction execution system, apparatus, or device and execute the instructions. In the context of this disclosure, a “computer-readable medium” is any tangible means that may contain or store the program for use by or in connection with the instruction execution system, apparatus, or device. The tangible computer readable medium may selectively be, for example, but is not limited to, an electronic, magnetic, optical, electromagnetic, or semiconductor system, apparatus or device. More specific examples, but nonetheless a non-exhaustive list, of tangible computer-readable media would include the following: a portable computer diskette (magnetic), a RAM (electronic), a read-only memory “ROM” (electronic), an erasable programmable read-only memory (EPROM or Flash memory) (electronic) and a portable compact disc read-only memory “CDROM” (optical). Note that the tangible computer-readable medium may even be paper (punch cards or punch tape) or another suitable medium upon which the instructions may be electronically captured, then compiled, interpreted or otherwise processed in a suitable manner if necessary, and stored in a computer memory.

The foregoing detailed description of one or more embodiments of the approach for meditation instruction that is institutionalized with employees and organizational leaders offered a type of meditation based upon the type of stress encountered and collects metrics associated with the meditation instruction has been presented herein by way of example only and not limitation. It will be recognized that there are advantages to certain individual features and functions described herein that may be obtained without incorporating other features and functions described herein. Moreover, it will be recognized that various alternatives, modifications, variations, or improvements of the above-disclosed embodiments and other features and functions, or alternatives thereof, may be desirably combined into many other different embodiments, systems or applications. Presently unforeseen or unanticipated alternatives, modifications, variations, or improvements therein may be subsequently made by those skilled in the art which are also intended to be encompassed by the appended claims. Therefore, the spirit and scope of any appended claims should not be limited to the description of the embodiments contained herein.

What is claimed is:

1. A method for providing meditation media to employees of an employer, comprising:
creating a user account for the employee that is a record in a database;
logging into the user account with a client;
presenting a stress survey at the client operated by the employee;
displaying a plurality of meditation media at the client in response to the stress survey;
streaming the meditation media from a streaming server in response to a selection of one of the plurality of meditation media at the client; and
recording that the selected streamed meditation media has been viewed in the user account.

2. The method of claim 1 where the streaming meditation media is viewed at the client.

3. The method of claim 1 where the streaming meditation media is viewed at a client separate from the client operated by the employee.

4. The method of claim 1 where the step of displaying the plurality of meditation media at the client in response to the stress survey further includes displaying various times for streaming the plurality of meditation media.

5. The method of claim 1 where the step of displaying a plurality of meditation media at the client in response to the stress survey further includes displaying various times and locations for streaming the multimedia.

6. A method for providing meditation media to employees of an employer, comprising:
creating a user account for the employee that is a record in a database;
logging into the user account with a client;
presenting a stress survey at the client operated by the employee;
displaying a plurality of meditation sessions at the client in response to the stress survey and allowing the user to select a meditation session to attend;
streaming meditation media from a streaming server in response to a selection of one of the plurality of meditation sessions at the client; and
recording information about the selected streamed meditation session when viewed by the user.

7. The method of claim 1 where the step of displaying a plurality of meditation media at the client in response to the stress survey further includes displaying various times and locations for streaming the meditation media.

8. The method of claim 1 where the streaming meditation media is viewed at the client.

9. The method of claim 1 where the streaming meditation media is viewed at client separate from the client operated by the employee.

10. A method for providing meditation media to employees of an employer, comprising:
creating a user account for the employee that is a record in a database;
logging into the user account with a client;
presenting a stress survey at the client operated by the employee;
displaying a plurality of meditation sessions at the client in response to the stress survey with various times and locations for streaming the meditation sessions and allowing the user to select a meditation session to attend;
streaming meditation media from a streaming server in response to a selection of one of the plurality of meditation sessions at a separate client at the selected location; and
recording information about the selected streamed meditation session when viewed by the user.
